(19) United States
(12) Patent Application Publication
(10) Pub. No.: US 2023/0090627 A1
(43) Pub. Date: Mar. 23, 2023

(54) Manufacturing Method For Flexible Tube For Endoscope, Endoscope Including Flexible Tube For Endoscope Manufactured Using Manufacturing Method For Flexible Tube For Endoscope, And Core Material Used In Manufacturing Flexible Tube For Endoscope

(71) Applicant: OLYMPUS MEDICAL SYSTEMS CORP., Tokyo (JP)

(72) Inventor: Kensaku HAYASHI, Tokyo (JP)

(73) Assignee: OLYMPUS MEDICAL SYSTEMS CORP., Tokyo (JP)

(21) Appl. No.: 17/940,413

(22) Filed: Sep. 8, 2022

(30) Foreign Application Priority Data
Sep. 13, 2021 (JP) 2021-148563

(51) Int. Cl. A61B 1/00; A61B 1/005

(52) U.S. Cl. CPC A61B 1/0011; A61B 1/005; A61B 1/00071

(57) ABSTRACT

A manufacturing method for a flexible tube for endoscope includes a step of extending a core material obtained by covering an outer circumference of a bar-like resin member with a first mesh tube in a major axis direction and winding a spiral tube around an outer circumference of the core material, a step of covering an outer circumference of the spiral tube with a second mesh tube, a step of molding an outer skin of a resin around an outer circumference of the second mesh tube and causing the resin to penetrate to a surface of the spiral tube, and a step of removing only the core material from a stacked tubular member formed by the core material, the spiral tube and the second mesh tube.

This application claims the benefit of Japanese Application No. 2021-148563 filed in Japan on Sep. 13, 2021, the contents of which are incorporated herein by this reference.

### BACKGROUND OF THE INVENTION

### 1. Field of the Invention

The present invention relates to a manufacturing method for a flexible tube for endoscope, an endoscope including the flexible tube for endoscope manufactured using the manufacturing method for the flexible tube for endoscope, and a core material used in the manufacturing of the flexible tube for endoscope.

### 2. Description of Related Art

In some flexible endoscope of related art, for example, a universal cord or a flexible tube section of an insertion section includes a flexible tube having flexibility. In general, a flexible tube for endoscope of this type is configured by, for example, a stacked tubular member in a form in which a spiral tube, a mesh tube, and an outer skin are stacked in this order from an inner circumferential side.

As a manufacturing method for the flexible tube for endoscope in the form of this type, various proposals have been made and put to practical use by, for example. Japanese Patent Publication No. 3490647.

In the manufacturing method for the flexible tube for endoscope disclosed by Japanese Patent Publication No. 3490647 or the like, the spiral tube is wound around the core material after an antifriction agent (powder of boron or the like) is applied to an outer circumferential surface of the core material. The antifriction agent is applied to improve workability, for example, at the time when the spiral tube is wound around an outer circumference of the core material or at the time when only the core material is finally removed from the stacked tubular member after the spiral tube, the mesh tube, and the outer skin are formed in order in the outer circumference of the core material.

### SUMMARY OF THE INVENTION

A manufacturing method for a flexible tube for endoscope in an aspect of the present invention includes: a step of, in a state in which a core material obtained by closely attaching a first mesh tube to and covering, with the first mesh tube, an outer circumference of a bar-like resin member having elasticity and stretchability is extended in a major axis direction, winding, around an outer circumference of the core material, a spiral tube formed by winding a metal band in a spiral shape; a step of covering an outer circumference of the spiral tube with a second mesh tube; a step of covering an outer circumference of the second mesh tube with resin to mold an outer skin; and a step of removing only the core material from a stacked tubular member formed by the core material and the spiral tube and the second mesh tube, an outer circumference of the stacked tubular member being covered by the outer skin.

An endoscope in an aspect of the present invention is an endoscope including an insertion section inserted into a subject, an operation section, and a universal cord, the endoscope including: a spiral tube formed by, in a state in which a core material obtained by closely attaching a first mesh tube to and covering, with the first mesh tube, an outer circumference of a bar-like resin member having elasticity and stretchability is extended in a major axis direction, winding a metal band around an outer circumference of the core material in a spiral shape; a second mesh tube covering an outer circumference of the spiral tube; and an outer skin molded by covering an outer circumference of the second mesh tube with resin, and a flexible tube manufactured using a manufacturing method for removing only the core material from a stacked tubular member formed by the core material and the spiral tube and the second mesh tube, an outer circumference of the stacked tubular member being covered by the outer skin, is applied to the insertion section or the universal cord.

A core material in an aspect of the present invention is a core material used in manufacturing of a flexible tube for endoscope obtained by stacking a spiral tube, a mesh tube, and a resin outer skin, the core material including: a bar-like resin member having elasticity and stretchability and having a predetermined length; and another mesh tube obtained by forming, in a tubular shape having a predetermined length, a metal net formed by weaving an element wire bundle obtained by bundling a plurality of metal element wires, the core material being formed by closely attaching the other mesh tube to and covering an outer circumference of the bar-like resin member.

Advantages of the present invention will be further clarified from the following detailed explanation.

### BRIEF DESCRIPTION OF THE DRAWINGS

FIG. 1 is an exterior view schematically showing an overall configuration of an endoscope system including an endoscope to which a flexible tube for endoscope manufactured by a manufacturing method for the flexible tube for endoscope in an embodiment of the present invention is applied;

FIG. 2 is a diagram showing a former half of a manufacturing process in the manufacturing method for the flexible tube for endoscope in the embodiment of the present invention;

FIG. 3 is a diagram showing a configuration of a core material used in flexible tube manufacturing by the manufacturing method for the flexible tube for endoscope in the embodiment of the present invention;

FIG. 4 is a diagram showing a third step of the manufacturing process in the manufacturing method for the flexible tube for endoscope in the embodiment of the present invention; and

FIG. 5 is a diagram showing the third step of the manufacturing process in the manufacturing method for the flexible tube for endoscope in the embodiment of the present invention.

### DETAILED DESCRIPTION OF THE PREFERRED EMBODIMENT

In general, the antifriction agent used in the manufacturing method for the flexible tube for endoscope disclosed by Japanese Patent Publication No. 3490647 described above is likely to scatter into a peripheral atmosphere during manufacturing and deteriorate a work environment. The antifriction agent is likely to adhere to a region other than a predetermined application region during manufacturing of the flexible tube. In such a case, work for cleaning or wiping off the antifriction agent adhering to the region other than the predetermined application region is necessary. Further, the antifriction agent is likely to remain on an inside of the flexible tube (for example, an inner side of the spiral tube). There is a risk that the antifriction agent remaining inside the flexible tube leaks to an outside in an assembly process for an endoscope and spoils appearance of a product. In such a case as well, work for, for example, wiping off the antifriction agent is necessary.

Incidentally, in the case of an endoscope of a so-called single use type that is discarded after being used only once, in general, assembly and the like are performed mainly under an environment in which cleanness is maintained such as a clean room in order to prevent, for example, mixing of foreign matters, bacteria and the like during manufacturing.

In this case, if the manufacturing method of the related art disclosed by Japanese Patent Publication No. 3490647 described above is applied, it is likely that the antifriction agent scatters into the peripheral atmosphere. Therefore, it is likely that a manufacturing environment is adversely affected, for example, cleanness of an atmosphere in the clean room or the like cannot be maintained.

Further, in the endoscope of the single use type, a configuration in which a flexible tube section, a universal cord, and the like are not formed in a watertight structure is sometimes adopted considering internal sterilization treatment and the like. In this case, if the antifriction agent or the like remains in or adheres to the inside of the flexible tube, it is likely that the antifriction agent or the like leaks to the outside at a transportation time or the like. Therefore, in the case of the endoscope of the single use type, it is demanded to avoid use of the antifriction agent or the like in a manufacturing process.

Accordingly, in order to manufacture the flexible tube without using the antifriction agent, it is conceivable to apply, as the core material, for example, a rigid bar-like member such as a metal material (for example, SUS) including a material having a smooth surface. However, in the core material made of such a rigid member, it is difficult to continuously mold an outer skin layer of the flexible tube in a state in which a plurality of core materials are coupled. As a result, productivity is deteriorated.

According to the present invention explained below, it is possible to provide a manufacturing method for a flexible tube for endoscope, an endoscope including the flexible tube for endoscope manufactured using the manufacturing method for the flexible tube for endoscope, and a core material used in the manufacturing of the flexible tube for endoscope that can maintain a work environment with high cleanness without using an antifriction agent and, at the same time, can secure internal cleanness of the flexible tube for endoscope after manufacturing completion, and can contribute to improvement of productivity.

The present invention is explained below according to an illustrated embodiment. Drawings used for the following explanation are schematically shown. In order to show respective components in recognizable sizes on the drawings, dimension relations, scales, and the like of respective members are sometime differentiated for each of the components and shown. Therefore, the present invention is not limited only to illustrated forms concerning the numbers of the respective components shown in the respective drawings, shapes of the respective components, ratios of sizes of the respective components, relative positional relations among the respective components, and the like.

First, before a manufacturing method for a flexible tube for endoscope in an embodiment of the present invention is explained, schematic configurations of an endoscope to which the flexible tube for endoscope manufactured by the manufacturing method is applied and an endoscope system including the endoscope are briefly explained below.

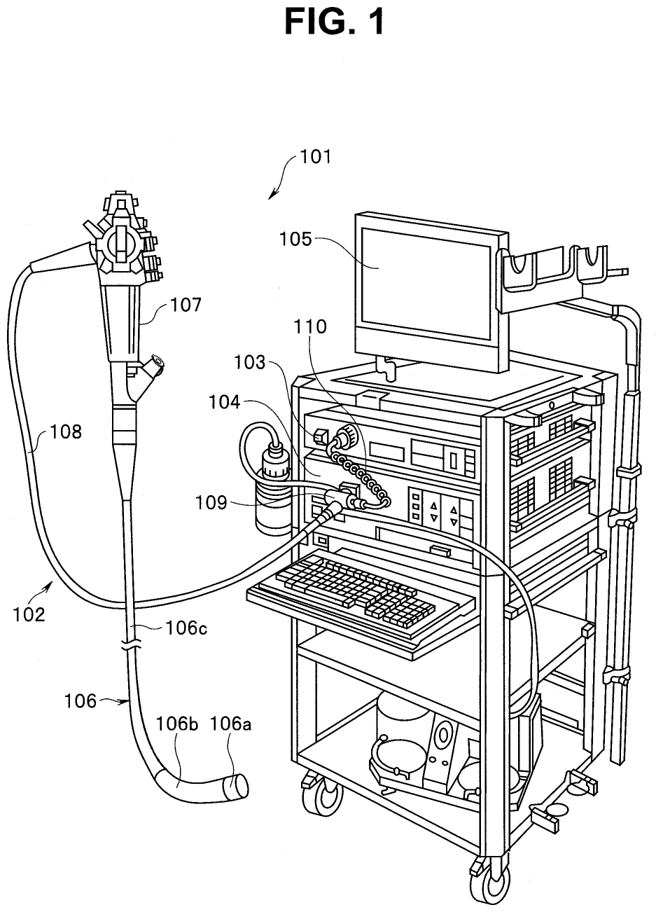

FIG. 1 is an exterior view schematically showing overall configurations of an endoscope to which a flexible tube for endoscope manufactured by a manufacturing method for the flexible tube for endoscope in an embodiment of the present invention is applied and an endoscope system including the endoscope. The endoscope system is basically substantially the same as an endoscope system having a general configuration in the past.

As shown in FIG. 1, an endoscope system 101 is mainly configured by an endoscope 102, a video processor 103, a light source apparatus 104, a monitor apparatus 105, and the like.

The endoscope 102 is an observation apparatus that observes an inside of a body cavity of a subject such as a living body and picks up an image of an inside of a body. The endoscope 102 includes an insertion section 106, an operation section 107 and a universal cord 108.

The insertion section 106 is a constituent unit configured by an elongated tube shape inserted into a body cavity or the like of a subject. The insertion section 106 includes a rigid distal end portion 106a provided on a distal end side, a bendable bending portion 106b provided at a rear end of the distal end portion 106a, and a flexible tube section 106c that is provided at a rear end of the bending portion 106b and is long and has flexibility.

The operation section 107 is a constituent unit that is disposed on a proximal end side of the insertion section 106 and in which various operation members that a surgeon grips to perform operation of the endoscope are provided.

The universal cord 108 is a constituent unit, one end of which is extended from a side portion of the operation section 107, configured by an elongated tube shape. A connector 109 is provided at the other end of the universal cord 108. The connector 109 is a connection member provided with an electric contact section and removably connected to the light source apparatus 104.

One end of a connection cable 110 is connected to a side surface of the connector 109. The other end of the connection cable 110 is connected to the video processor 103.

An image pickup device, an illumination apparatus, and the like are provided at the distal end portion 106a of the insertion section 106. A signal line, a light guide fiber, and the like extended from the image pickup device, the illumination apparatus, and the like are inserted through insides of the insertion section 106, the operation section 107 and the universal cord 108 and connected to the light source apparatus 104 and the video processor 103 through the connector 109 and the connection cable 110.

The video processor 103 is a processor that receives an image pickup signal from the endoscope 102 and applies predetermined image processing to the image pickup signal. The video processor 103 is connected to the monitor apparatus 105 by a not-shown connection cable. Consequently, an image signal for display subjected to predetermined image processing by the video processor 103 is outputted to the monitor apparatus 105. The monitor apparatus 105 receives the image signal for display and displays an image of an inside of a body cavity acquired by the endoscope 102.

The light source apparatus 104 is an apparatus that supplies illumination light for illuminating a subject. The illumination light supplied from the light source apparatus 104 is transmitted to the distal end portion 106a of the insertion section 106 through the light guide fiber inserted through the connector 109, the universal cord 108, the operation section 107, and the insertion section 106. The illumination light is irradiated from a front surface of the distal end portion 106a toward the subject.

In the endoscope 102 included in the endoscope system 101 having such a configuration, the flexible tube section 106c of the insertion section 106 and the universal cord 108 are formed as an elongated tube shape that is long and has flexibility. In this case, in order to protect a signal cable and the like inserted through the insides of the flexible tube section 106c and the universal cord 108 while guaranteeing flexibility of the flexible tube section 106c and the universal cord 108, a flexible tube for endoscope is applied to the flexible tube section 106c and the universal cord 108 in the endoscope 102.

In general, the flexible tube for endoscope has a form in which a spiral tube, a mesh tube, and an outer skin are stacked and formed in a tubular shape. In this case, the spiral tube is formed in a tubular shape by winding, in a spiral shape, a thin plate-like member made of metal, formed in a belt shape, and having elasticity. The mesh tube is obtained by forming, in a tubular shape, a metal net formed by weaving an element wire bundle obtained by bundling a plurality of metal element wires and is wound around an outer circumference of the spiral tube. The outer skin is formed by solidifying, for example, a resin material and covers an outer circumferential surface of the mesh tube.

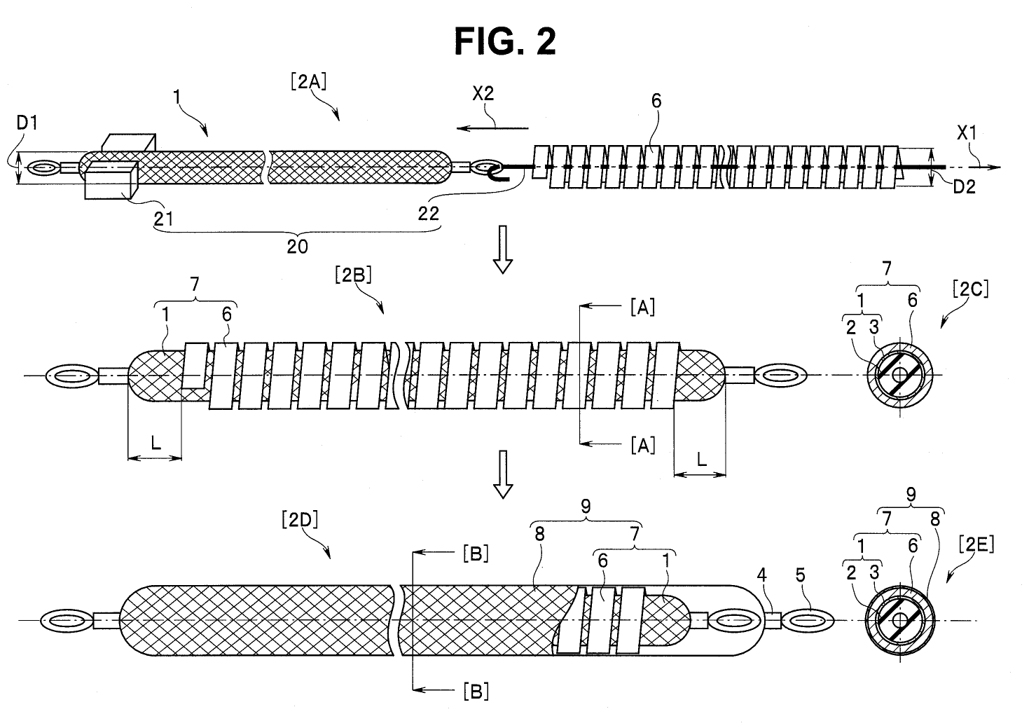

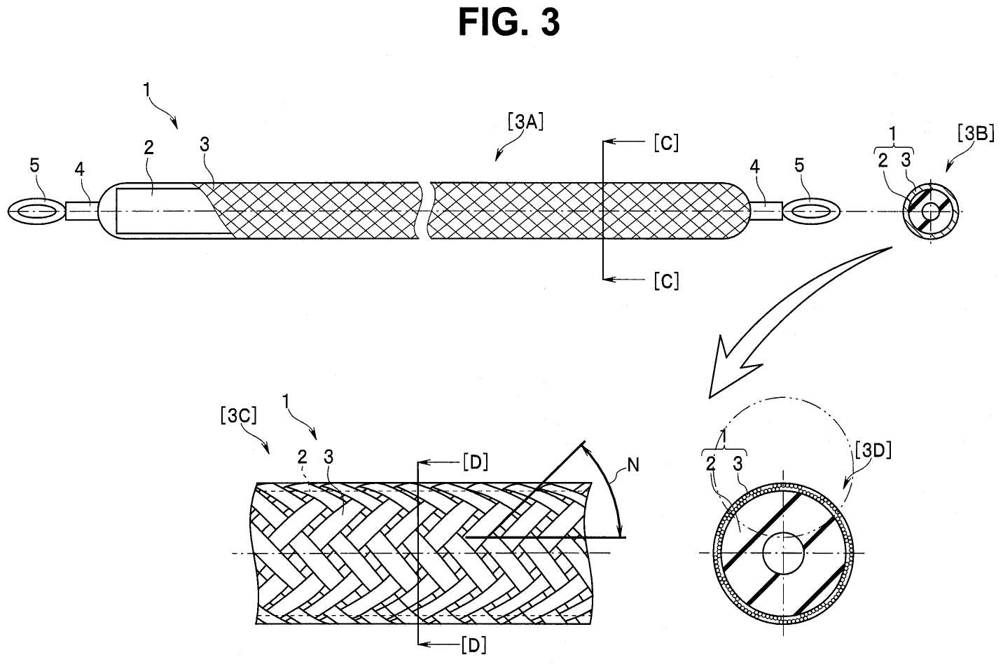

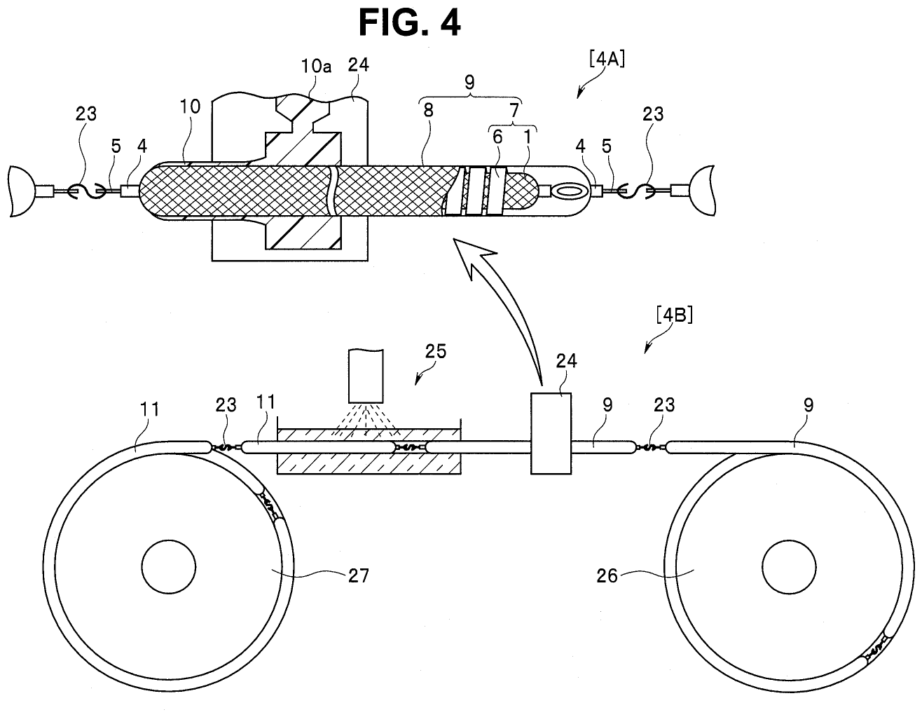

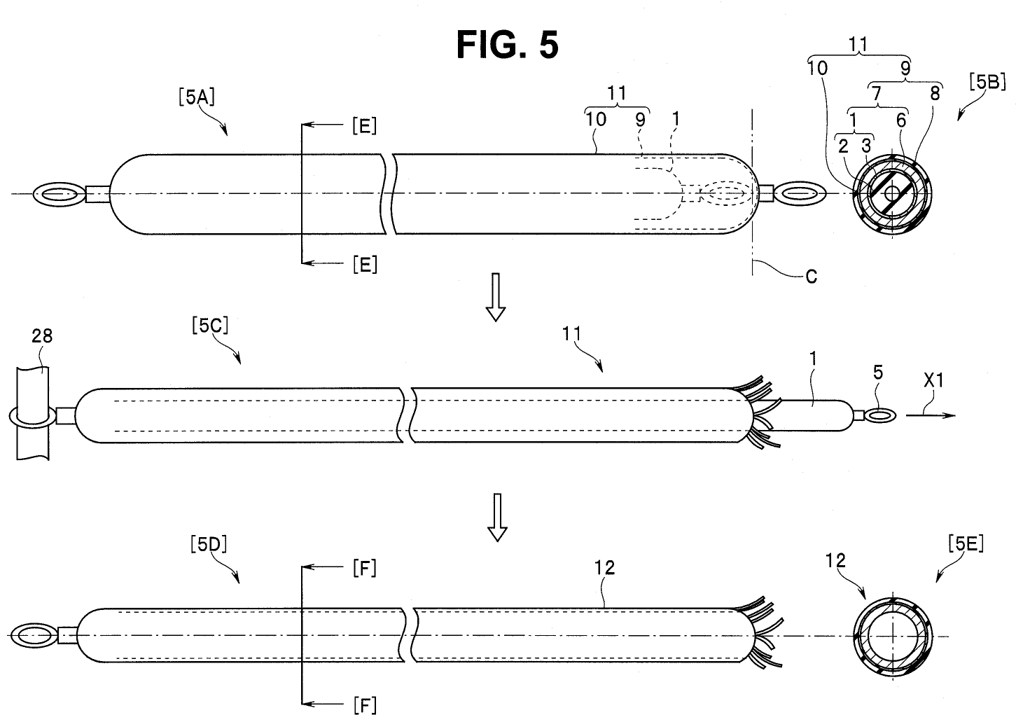

Subsequently, a manufacturing method for a flexible tube for endoscope in the embodiment of the present invention is explained below with reference to FIGS. 2 to 5. FIGS. 2 to 5 are diagrams for explaining the manufacturing method for the flexible tube for endoscope in the embodiment of the present invention. FIG. 2 shows a former half of a manufacturing process in the manufacturing method for the flexible tube for endoscope in the present embodiment. FIG. 3 is a diagram showing a configuration of a core material used in flexible tube manufacturing by the manufacturing method for the flexible tube for endoscope in the present embodiment. FIGS. 4 and 5 are diagrams showing a third step of the manufacturing process in the manufacturing method for the flexible tube for endoscope in the present embodiment.

First, in FIG. 2, a sign [2A] shows a first step in the manufacturing method for the flexible tube for endoscope in the present embodiment.

The first step is a step of winding a spiral tube 6 around an outer circumference of a core material 1 extended in a major axis direction.

Here, the core material 1 is a bar-like member that has a predetermined length set slightly longer than length of a flexible tube for endoscope (see FIG. 1) to be manufactured and around an outer circumferential surface of which the spiral tube 6 is wound in the first step in the manufacturing method. In other words, the core material 1 is a member functioning as a base for defining a shape of the flexible tube to be manufactured.

As shown in FIG. 3, the core material 1 is mainly configured by a bar-like resin member 2, a first mesh tube 3, splice bands 4, and coupling rings 5.

Note that, in FIG. 3, a sign [3A] shows a plane of the core material. A sign [3B] in FIG. 3 shows a cross section taken along a [C]-[C] line. A sign [3C] in FIG. 3 is a diagram showing a surface of the core material in detail. A sign [3D] in FIG. 3 shows an enlarged cross section (a cross section taken along a [D]-[D] line) of the core material.

The bar-like resin member 2 has elasticity and stretchability, and is made of a resin member (for example, silicon rubber, fluorocarbon rubber, or a synthetic resin material) formed in a bar shape (for example, a columnar shape or a cylindrical shape) as a whole.

Note that the bar-like resin member 2 is more desirably a material further having heat resistance. In other words, in the third step explained below, an outer circumference of a second intermediate manufactured product 9 is covered by thermoplastic resin 10a in a heat-melted state (hereinafter simply referred to as resin 10a). At this time, the core material 1 is disposed on an inside of the second intermediate manufactured product 9. When an outer circumference of the second intermediate manufactured product 9 is covered by an outer skin 10 using an extrusion molding machine 24, the resin 10a heat-melted at, for example, approximately 250 degrees Celsius adheres to the outer circumference of the second intermediate manufactured product 9. The heat is likely to be transmitted to the bar-like resin member 2 of the core material 1 on the inside. Therefore, considering this, the bar-like resin member 2 of the core material 1 is desirably a material having predetermined heat resistance.

The first mesh tube 3 is obtained by, for example, forming, in a tubular shape, a metal net formed by weaving an element wire bundle obtained by bundling a plurality of metal element wires.

The core material 1 is formed by closely attaching the first mesh tube 3 to and covering, with the first mesh tube 3, an outer circumference of the bar-like resin member 2. In this case, the first mesh tube 3 is narrowed at both end portions of the core material 1 and fixed by the splice bands 4. The coupling rings 5 are provided at both the end portions of the core material 1. The coupling rings 5 are used to couple a plurality of core materials 1 in the second step or the third step (explained below) in the manufacturing method for the endoscope flexible tube in the present embodiment.

Note that the core material 1 is formed to be slightly longer than length of the spiral tube 6. For example, total length of the core material 1 is set approximately 50 to 100 mm longer than the spiral tube 6. In other words, as shown in FIG. 2, when the spiral tube 6 is wound around the core material 1, both the end portions of the core material 1 are respectively exposed from both ends of the spiral tube 6 by length of a degree indicated by a sign L in FIG. 2. In this case, it is desirable to set L to approximately 25 to 50 mm.

An outer diameter of the core material 1 is set substantially equal to or slightly larger than an inner diameter of the spiral tube 6. For example, as shown in FIG. 2, when the outer diameter of the core material 1 is represented by a sign D1 and the inner diameter of the spiral tube 6 is represented by a sign D2, the outer diameter and the inner diameter are set in a relation of

D1≥D2

when both of the core material 1 and the spiral tube 6 are in a natural state.

The first mesh tube 3 in the core material 1 is preferably set in a range of a braiding angle N=40° to 70° shown in FIG. 3. Further, the first mesh tube 3 in the core material 1 is more preferably set in a range of a braiding angle N=50° to 65°.

In the first step in the manufacturing method for the flexible tube for endoscope in the present embodiment performed using the core material 1 configured as explained above, the spiral tube 6 is wound around an outer circumference of the core material 1 and disposed. The spiral tube 6 is formed in a tubular shape by winding a metal band in a spiral shape.

First, as indicated by a sign [2A] in FIG. 2, the core material 1 is fixed to a fixing table 21 (for example, a vise) of a fixing tensile jig 20. A wire 22 is coupled to the coupling ring 5 of the core material 1 fixed to the fixing table 21. At this time, the core material 1 and the wire 22 are disposed with axial directions thereof aligned. The wire 22 is inserted through the spiral tube 6. Note that the spiral tube 6 is formed in a tube shape in advance.

When the wire 22 is pulled in an arrow X1 direction in FIG. 2 in the axial direction in this state, the core material 1 changes to a reduced-diameter state. In this case, the braiding angle of the first mesh tube 3 of the core material 1 fluctuates according to the pulling and compression. Accordingly, even the core material 1 in which an outer circumference of the bar-like resin member 2 is covered by the first mesh tube 3 is capable of extending and contracting in the axial direction.

When the core material 1 is reduced in diameter by the wire 22 in this way, the outer diameter of the core material 1 is smaller than the inner diameter of the spiral tube 6 (D1<D2). The spiral tube 6 is moved in an arrow X2 direction in FIG. 3 and disposed in a predetermined position in the outer circumference of the core material 1 while the reduced-diameter state of the core material 1 is maintained.

When the spiral tube 6 is disposed in the predetermined position in the outer circumference of the core material 1 in this way, a tensile force of the core material 1 is released. Consequently, the reduced-diameter state of the core material 1 returns to a normal state. Consequently, the core material 1 returns from the reduced-diameter state to the normal state indicated by the outer diameter D1.

As explained above, when both of the core material 1 and the spiral tube 6 are in the natural state, D1≥D2. The core material 1 has elasticity and stretchability.

Accordingly, the spiral tube 6 having a small diameter with respect to the core material 1 shown in FIG. 2 is surely fixed in the outer circumference of the core material 1.

With such a first step, a first intermediate manufactured product 7 shown in signs [2B] and [2C] in FIG. 2 is manufactured. The sign [2B] in FIG. 2 shows a plane of the first intermediate manufactured product 7. The sign [2C] in FIG. 2 shows a cross section of the first intermediate manufactured product 7 taken along a [A]-[A] line. Note that the first intermediate manufactured product 7 is a bar-like member in a state in which the spiral tube 6 is wound around the outer circumference of the core material 1 and disposed in the predetermined position.

Subsequently, the second step in the manufacturing method for the flexible tube for endoscope in the present embodiment is performed. In FIG. 2, signs [2D] and [2E] show a second intermediate manufactured product 9 manufactured by the second step in the manufacturing method for the flexible tube for endoscope in the present embodiment.

The second step is a step of winding a second mesh tube 8 around an outer circumference of the first intermediate manufactured product 7 manufactured by the first step (that is, the outer circumference of the spiral tube 6) and manufacturing the second intermediate manufactured product 9.

Like the first mesh tube 3, the second mesh tube 8 is obtained by, for example, forming, in a tubular shape, a metal net formed by weaving an element wire bundle obtained by bundling a plurality of metal element wires.

In the second step, the outer circumference of the first intermediate manufactured product 7 (the spiral tube 6) is covered by the second mesh tube 8 and disposed. Both end portions of the second mesh tube 8 are fixed using the splice band 4 in a narrowed state. The coupling rings 5 are formed at the end portions.

With such a second step, the second intermediate manufactured product 9 shown in the signs [2D] and [2E] in FIG. 2 is manufactured. The sign [2D] in FIG. 2 shows a plane of the second intermediate manufactured product 9. The sign [2E] in FIG. 2 shows a cross section of the second intermediate manufactured product 9 taken along a [B]-[B] line. Note that the second intermediate manufactured product 9 is a bar-like member in a state in which the outer circumference of the first intermediate manufactured product 7 (the core material 1 and the spiral tube 6) is covered by the second mesh tube 8.

Subsequently, the third step in the manufacturing method for the flexible tube for endoscope in the present embodiment is performed. FIG. 4 shows the third step in the manufacturing method for the flexible tube for endoscope in the present embodiment. Note that, in FIG. 4, a sign [4A] enlarges and shows a step of molding the outer skin 10. In FIG. 4, a sign [4B] schematically shows the entire third step.

In FIG. 5, signs [5A] and [5B] show a third intermediate manufactured product 11 manufactured by the third step in the manufacturing method for the flexible tube for endoscope in the present embodiment.

The third step is a step of covering, with the extrusion molding in FIG. 4, the outer circumference of the second intermediate manufactured product 9 manufactured by the second step (that is, an outer circumference of the second mesh tube 8) with the resin 10a to form the outer skin 10 and manufacturing the third intermediate manufactured product 11. The third step also includes a step of cooling and solidifying the formed outer skin 10.

The outer skin 10 is formed by cooling and solidifying the resin 10a. In the third step, the outer circumference of the second mesh tube 8 is covered by the outer skin 10 using a well-known extrusion molding machine 24.

The outer circumference of the second intermediate manufactured product 9 (the second mesh tube 8) is covered by the outer skin 10 made of, for example, thermoplastic resin as explained below. First, the first step to the third step explained above are repeated to manufacture a plurality of second intermediate manufactured products 9 in advance.

For example, hook members 23 formed in an S shape or a C shape are hooked on the coupling rings 5 respectively formed at the respective both end portions of the plurality of second intermediate manufactured products 9 and a large number of the second intermediate manufactured products 9 are coupled to form a long second intermediate manufactured product 9. The long second intermediate manufactured product 9 obtained by coupling the large number of the second intermediate manufactured products 9 is wound around a supply drum 26.

The second intermediate manufactured product 9 wound around the supply drum 26 is drawn out from one end portion and inserted through the extrusion molding machine 24 and a well-known cooling apparatus 25 (in FIG. 4, a water cooling-type apparatus is illustrated). Consequently, the outer circumference of the second intermediate manufactured product 9 is continuously covered by the outer skin 10. In this way, when the outer circumference of the second intermediate manufactured product 9 is covered by the outer skin 10, the outer skin 10 is continuously molded by covering the plurality of second intermediate manufactured products 9. Thereafter, the third intermediate manufactured product 11 on which the outer skin 10 is molded is wound by a winding drum 27 in a state in which a plurality of third intermediate manufactured products 11 are coupled. The hook members 23 are removed from the third intermediate manufactured product 11 wound by the winding drum 27 and the third intermediate manufactured product 11 is separated into single third intermediate manufactured products 11 to be supplied to a next step.

In this case, in the third step, an outer surface portions of the hook members 23 may also be covered by the resin 10a. However, when the third intermediate manufactured product 11 is separated into the individual third intermediate manufactured products 11, the resin 10a in coupling portions only has to be peeled off.

With such a third step, the third intermediate manufactured product 11 shown in the signs [5A] and [5B] in FIG. 5 is manufactured. The sign [5A] in FIG. 5 shows a plane of the third intermediate manufactured product 11. The sign [5B] in FIG. 5 shows a cross section of the third intermediate manufactured product 11 taken along an [E]-[E] line. Note that the third intermediate manufactured product 11 is a stacked tubular member in a state in which the outer circumference of the second intermediate manufactured product 9 (the core material 1, the spiral tube 6, and the second mesh tube 8) is covered by the outer skin 10.

Note that, in the second step explained above, substantially the same step form as the third step can be adopted. In other words, in the second step, work for coupling the plurality of first intermediate manufactured products 7 using the hook members 23 hooked on the respective coupling rings 5 to form the long first intermediate manufactured product 7 and covering the individual first intermediate manufactured products 7 with the second mesh tube 8 can be continuously performed.

Subsequently, a fourth step in the manufacturing method for the flexible tube for endoscope in the present embodiment is performed. In FIG. 5, a sign [5C] shows the fourth step. Note that, in FIG. 5, signs [5D] and [5E] show a flexible tube for endoscope 12 serving as a final manufactured product in the manufacturing method for the flexible tube for endoscope in the present embodiment.

The fourth step is a step of removing only the core material 1 from the third intermediate manufactured product 11 (the stacked tubular member including the core material 1, the spiral tube 6, the second mesh tube 8, and the outer skin 10) manufactured by the third step.

In the fourth step, first, one end portion of the third intermediate manufactured product 11 is cut to expose one end portion of the core material 1 to the outside. In an example shown in FIG. 5, a cut region is indicated by an alternate long and two short dashes line indicated by a sign C.

The other end portion of the third intermediate manufactured product 11 is fixed using a predetermined fixing jib 28. In this state, the coupling ring 5 of the core material 1 exposed to the outside of the one end portion of the third intermediate manufactured product 11 is pulled in an arrow X1 direction shown in FIG. 5. Then, the core material 1 is extended to be reduced in diameter by elasticity and stretchability of the core material 1. Consequently, the core material 1 has a smaller diameter than the inner diameter of the spiral tube 6 in the third intermediate manufactured product 11. Therefore, the core material 1 can be easily pulled out. The pulled-out core material 1 can be reused at a next manufacturing time.

With such a fourth step, the flexible tube for endoscope 12 serving as the final manufactured product shown in the signs [5D] and [5E] in FIG. 5 is manufactured. The sign [5D] in FIG. 5 shows a plane of the flexible tube for endoscope 12. The sign [5E] in FIG. 5 shows a cross section of the flexible tube for endoscope 12 taken along an [F]-[F] line.

The flexible tube 12 of an endoscope insertion section manufactured in this way is cut into a predetermined length and, thereafter, subjected to predetermined processing for both end portions and assembled as a flexible tube section or a universal cord of an endoscope.

Note that the manufacturing method in the embodiment is mainly used in a clean room in which air cleanness is secured. The flexible tube for endoscope 12 manufactured by the manufacturing method is applied to a flexible tube section or a universal cord in a reuse-type endoscope usually usable a plurality of times. The flexible tube for endoscope 12 can also be applied to a flexible tube section or a universal cord in a single use endoscope that has the same structure as the reuse-type endoscope and, for example, is discarded after being used only once.

As explained above, according to the embodiment, it is possible to present a manufacturing method for manufacturing, using the core material 1 obtained by closely attaching the first mesh tube 3 to and covering, with the first mesh tube 3, the outer circumference of the bar-like resin member 2 having elasticity, stretchability, and heat resistance, the flexible tube for endoscope 12 including a tubular member of a three-layer structure of the spiral tube 6, the second mesh tube 8, and the outer skin 10. In the manufacturing method, it is unnecessary to use an antifriction agent used in the past. Therefore, it is possible to maintain high cleanness in a work environment (an internal environment such as a clean room). At the same time, it is possible to secure internal cleanness of the flexible tube for endoscope 12 after manufacturing completion. Work for cleaning or wiping off the antifriction agent adhering to a product is unnecessary. It is possible to contribute to improvement of productivity.

Further, since the core material 1 is configured using the material having elasticity and stretchability, in the third step of covering the outer circumference of the second intermediate manufactured product 9 with the outer skin 10, the long second intermediate manufactured product 9 obtained by coupling the plurality of second intermediate manufactured products 9 can be wound around the supply drum 26 and the third intermediate manufactured product 11 on which the outer skin 10 is molded can be wound around the winding drum 27. Consequently, it is possible to continuously mold the outer skin 10 to cover the plurality of second intermediate manufactured products 9. Therefore, it is possible to manufacture the flexible tube for endoscope 12 at high productivity.

Note that, in the second step as well, it is possible to contribute to further improvement of productivity by coupling and treating a plurality of first intermediate manufactured products 7.

In the first step of winding the spiral tube 6 around the outer circumference of the core material 1 and disposing the spiral tube 6 and the fourth step of removing only the core material 1 from the third intermediate manufactured product 11, the core material 1 is pulled to be reduced in diameter. Consequently, it is possible to extremely easily dispose the spiral tube 6 in the outer circumference of the core material 1 and removing the core material 1 from the third intermediate manufactured product 11. In any case, the spiral tube 6 is not disordered in alignment or damaged by a frictional force that occurs between the core material 1 and the spiral tube 6.

The manufacturing method in one embodiment of the present invention can be applied to the endoscope of the related art. However, the manufacturing method of the present invention is not limited to this and can be applied to a single use-type endoscope as well.

The present invention is not limited to the embodiment explained above. It goes without saying that various modifications and applications can be implemented within a range not departing from the gist of the invention. Further, inventions in various stages are included in the embodiment. Various inventions can be extracted according to appropriate combinations in a disclosed plurality of constituent elements. For example, when the problems to be solved by the invention can be solved and the effects of the invention can be obtained even if several constituent elements are deleted from all the constituent elements described in the embodiment, a configuration in which the constituent elements are deleted can be extracted as an invention. Further, constituent elements described in different embodiments may be combined as appropriate. The present invention is not limited by a specific implementation mode of the present invention except that the present invention is limited by the appended claims.

What is claimed is:

1. A manufacturing method for a flexible tube for endoscope comprising:
a step extending a core material in a longitudinal direction, wherein the core material is formed by coating a first mesh tube in close contact with an outer circumference of a bar-like resin member having elasticity and stretchability;
a step of winding a spiral tube formed by winding a metal band around an outer circumference of the core material;
a step of covering an outer circumference of the spiral tube with a second mesh tube;
a step of covering an outer circumference of the second mesh tube with resin to mold an outer skin; and
a step of removing only the core material from a stacked tubular member formed by the core material and the spiral tube and the second mesh tube, an outer circumference of the stacked tubular member being covered by the outer skin.

2. The manufacturing method for the flexible tube for endoscope according to claim 1, wherein the outer skin is made of resin molded by covering the outer circumference of the second mesh tube by extrusion molding.

3. The manufacturing method for the flexible tube for endoscope according to claim 1, wherein the bar-like resin member further has heat resistance.

4. The manufacturing method for the flexible tube for endoscope according to claim 1, wherein a braiding angle of the first mesh tube is within a range of an angle of 40 degrees to 70 degrees.

5. The manufacturing method for the flexible tube for endoscope according to claim 4, wherein the braiding angle of the first mesh tube is further within a range of an angle of 50 degrees to 65 degrees.

6. An endoscope including an insertion section inserted into a subject, an operation section, and a universal cord, the endoscope comprising:
a spiral tube formed by, in a state of extending a core material in a longitudinal direction, the core material being obtained by coating a first mesh tube in close contact with an outer circumference of a bar-like resin member having elasticity and stretchability, winding a metal band around an outer circumference of the core material;
a second mesh tube covering an outer circumference of the spiral tube; and
an outer skin molded by covering an outer circumference of the second mesh tube with resin, wherein
a flexible tube manufactured using a manufacturing method for removing only the core material from a stacked tubular member formed by the core material and the spiral tube and the second mesh tube, an outer circumference of the stacked tubular member being covered by the outer skin, is applied to the insertion section or the universal cord.

7. The endoscope according to claim 6, wherein the flexible tube is applied to a flexible tube section of the endoscope.

8. The endoscope according to claim 6, wherein the flexible tube is applied to the universal cord.

9. The endoscope according to claim 6, wherein
the flexible tube is manufactured in a clean room in which air cleanness is secured, and
the endoscope is a single use endoscope that is discarded after being used only once.

10. A core material used in manufacturing of a flexible tube for endoscope obtained by stacking a spiral tube, a mesh tube, and a resin outer skin, the core material comprising:
a bar-like resin member having elasticity and stretchability and having a predetermined length; and
another mesh tube obtained by forming, in a tubular shape having a predetermined length, a metal net formed by weaving an element wire bundle obtained by bundling a plurality of metal element wires,
the core material being formed by closely attaching the other mesh tube to and covering, with the other mesh tube, an outer circumference of the bar-like resin member.

11. The core material according to claim 10, wherein a ring-like coupling member is fixed to at least one end in an axial direction.
